(19) United States
(12) Patent Application Publication
(10) Pub. No.: US 2007/0073334 A1
(43) Pub. Date: Mar. 29, 2007

(54) Combined electrolytic and mechanical separation background

(76) Inventor: Kamal Ramzipoor, Fremont, CA (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/238,399

(22) Filed: Sep. 29, 2005

(51) Int. Cl. A61M 29/00

(52) U.S. Cl. 606/200

(57) ABSTRACT

Apparatus and method are disclosed by which a distal end portion of an elongate member may be deposited at selected sites in body passageways. The apparatus includes an elongate member having a detachable member for delivery to a target location. The apparatus also has a detachment zone adjacent the distal end section for rupturing when a combination of electrolytic and mechanical energy is applied. The detachment zone may include any of a broad variety or materials and/or structures, but is configured and/or adapted to rupture and/or break through the use of a combination of electrolytic and mechanical energy, thus achieving detachment of the detachable member. A catheter or other delivery device can be coupled to or surrounds the apparatus for delivery to a target location.

### TECHNICAL FIELD

The present invention relates generally to medical devices for delivering a detachable member to a target location within the anatomy of a patient. More specifically, the present invention relates to a method and apparatus for delivering a device and/or member that is detachably attached to an elongate member into body cavities or passageways, and detaching the device and/or member from the elongate member using a combination of electrolytic and mechanical energy.

### BACKGROUND

The delivery of a detachable medical device within the anatomy of a patient is generally known. For example, vaso-occlusive devices or implants are used for a wide variety of reasons. They are often used for treatment of intra-vascular aneurysms. This is to say that the treatment involves the placement of a vaso-occlusive device in an aneurysm to cause the formation of a clot and eventually of a collagenous mass containing the vaso-occlusive device. This seals and/or fills the aneurysm thereby preventing the weakened wall of the aneurysm from being exposed to the pulsing blood pressure of the open vascular lumen. Treatment of aneurysms in this fashion is a significant improvement over the surgical method typically involved.

A variety of methods, structures, and assemblies have been developed for delivering detachable medical devices within the anatomy of a patient, each having certain advantages and disadvantages. However, there is an ongoing need to provide alternatives.

### SUMMARY OF SOME EXAMPLE EMBODIMENTS

The invention provides several alternative structures, assemblies, devices, and/or methods for delivering a detachable member to a target location within the anatomy of a patient. Some embodiments include an apparatus and/or method for quickly and reliably detaching a selectively detachable member or element from the distal end of an elongated member in a body passageway by the use of a combination of electrolytic and mechanical detachment energies and/or means.

For example, some embodiments relate to a method a method of disposing a detachable member at a target location in the anatomy of a patient. An elongate member can be provided having a detachable member at a distal end, wherein the detachable member is selectively detachable from the elongate member at a detachment zone by the use of a combination of electrolytic and mechanical energy. The elongate member can be inserted into the body passageway until the detachable member is disposed at the target location. A current can be delivered to the elongate member to initiate electrolytic degradation within the detachment zone, and a mechanical force can be delivered to the elongate member, thereby detaching the detachable member at the detachment zone. The elongate member can then be withdrawn the from the body passageway. In some embodiments, the elongate member has a power generator attached to a proximal end for the delivery of electrical current for electrolytic degradation. In some embodiments, a power generator may be used to deliver the mechanical force.

Some example embodiments relate to a medical apparatus having a detachable member for delivery to a target location within the anatomy of a patient. The apparatus may include an elongate member having a proximal end and a distal end, and a detachable member disposed at the distal end of the elongate member. A detachment zone may be disposed adjacent the detachable member, wherein the detachment zone can be made of a material susceptible to electrolytic degradation. Also, the detachment zone may include structure susceptible to mechanical separation upon the application of a mechanical force after electrolytic degradation of the detachment zone, such that the detachable member is detached from the elongate member by applying both electrolytic and mechanical forces.

Yet some further example embodiments relate to a device for disposing a detachable member at a target location in a body passageway. The device can include an elongate member having a proximal portion and a distal portion, and a detachment zone interconnecting the proximal and distal portions. The device can also include first means for selectively degrading the detachment zone, and second means for selectively detaching the distal portion, wherein the distal portion is detached by activating both the first and second means for selectively detaching the distal portion.

The above summary of some embodiments is not intended to describe each disclosed embodiment or every implementation of the invention. The figures, and detailed description which follow, more particularly exemplify the embodiments. Other objects, features and advantages of the invention will become apparent from a consideration of the following detailed description presented in connection with the accompanying claims and drawings.

### BRIEF DESCRIPTION OF THE DRAWINGS

The invention may be more completely understood in consideration of the following description of various embodiments, in connection with the accompanying drawings, in which:

FIG. 1 shows a fragmented side view of an assembly and/or device including an elongated member and a detachable member in accordance with an example embodiment;

FIG. 2A shows a fragmented side, partial cross-sectional view of the assembly and/or device of FIG. 1 inserted within the anatomy of a patient with the aid of a delivery device, such as a catheter;

FIG. 2B is a view similar to that shown in FIG. 2A showing the device after the initiation of electrolytic degradation of the detachment zone;

FIG. 2C is a view similar to that shown in FIG. 2B showing the device after the initiation of mechanical vibrational energy to cause detachment at the detachment zone;

FIG. 3 shows a side, fragmented, cross-sectional view of a device made in accordance with another embodiment similar to that shown in FIG. 1, but including an insulating layer;

FIG. 4A shows a fragmented side view of an alternative embodiment of an assembly and/or device including an alternative construction of the detachment zone;

FIG. 4B shows a fragmented side view of an alternative embodiment of an assembly and/or device including an alternative construction of the detachment zone;

FIG. 5A shows a fragmented side view of an alternative embodiment of an assembly and/or device including an alternative construction of the detachment zone;

FIG. 5B shows a fragmented side view of an alternative embodiment of an assembly and/or device including an alternative construction of the detachment zone;

FIG. 6 shows a fragmented side view of an alternative embodiment of an assembly and/or device including an alternative construction of the detachment zone;

FIG. 7 shows a fragmented side view of an alternative embodiment of an assembly and/or device including an alternative construction of the detachment zone;

FIG. 8 shows a fragmented side view of an alternative embodiment of an assembly and/or device including an alternative construction of the detachment zone; and

FIG. 9 shows a fragmented partial cross-sectional side view of an alternative embodiment of an assembly and/or device including an alternative construction of the detachment zone.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings, in which like elements in different drawings are numbered in like fashion. The drawings, which are not necessarily to scale, depict selected embodiments and are not intended to limit the scope of the invention. Although examples of construction and materials may be described for some elements, those skilled in the art will recognize that many of the examples provided have suitable alternatives that may be utilized. The following description relates particularly to an illustrative vaso-occlusion device wherein a detachable vaso-occlusive member can be delivered to a target location within the vasculature of a patient, for example, to treat an aneurism, or the like. However, it should be recognized that other embodiments may be used to deliver other types of devices, such as other occlusing devices, stents, filters, drug delivery devices, or the like, to locations within other body passageways, as desired.

At least some embodiments relate to the use of a combination of electrolytic and mechanical vibrational detachment methods, techniques, forces, and/or structures for providing or delivering a detachable medical device or member in the anatomy of a patient. For example, a medical device or assembly can include a detachable device or member detachably disposed on an elongated member that can be used in delivering the detachable member to the target location within the anatomy. The detachable disposition between the elongated member and the detachable member can include structure and/or material such that once disposed at the target location within the anatomy, the detachable member can be selectively detached or separated from the elongated member through the use of a combination of both electrolytic and mechanical vibrational energy. As such, the detachable member can be detached and remain or be left in the anatomy, as desired, while the elongated delivery member can be withdrawn, as desired.

For example, a detachment zone, region, and/or structure can be defined within and/or between the elongated member and the detachable member. The detachment zone can include structure and/or material that is susceptible to electrolytic degradation in the body when electrical current is applied thereto. As a result, the mass, structure, and/or integrity within the detachment zone can be reduced using electrolytic degradation—thereby rendering the detachment zone more susceptible to detachment by the application of mechanical vibrational energy. Furthermore, the detachment zone or region can include structure and/or material that may be susceptible to detachment when mechanical vibrational energy is applied thereto. In at least some embodiments, the structure and/or method can be such that the detachment zone can be electrolytically degraded to weaken the detachment zone, and the detachable member can be detached and/or separated from the elongated member through the use of mechanical vibrational energy that is applied to the weakened detachment zone. The electrolytic energy and mechanical vibrational forces used to achieve the detachment and/or separation can be applied sequentially and/or simultaneously.

The use of a combination of both electrolytic degradation and mechanical vibrational forces to achieve separation and/or detachment of the detachable member may provide certain advantages in at least some embodiments. For example, in some instances, a rapid, reliable method for delivering the detachable device and/or member within the anatomy of the patient can be achieved. For example, in some embodiments, the medical device or assembly can include structure or materials within the detachment zone that is relatively hearty, for example, to reduce the likelihood of premature or unwanted detachment of the detachable member during navigation of the device through the anatomy to a target location. However, when detachment is desired, the use of a combination of both electrolytic degradation to weaken the detachment zone, and mechanical vibrational forces to achieve detachment can allow for relatively reliable and quick detachment.

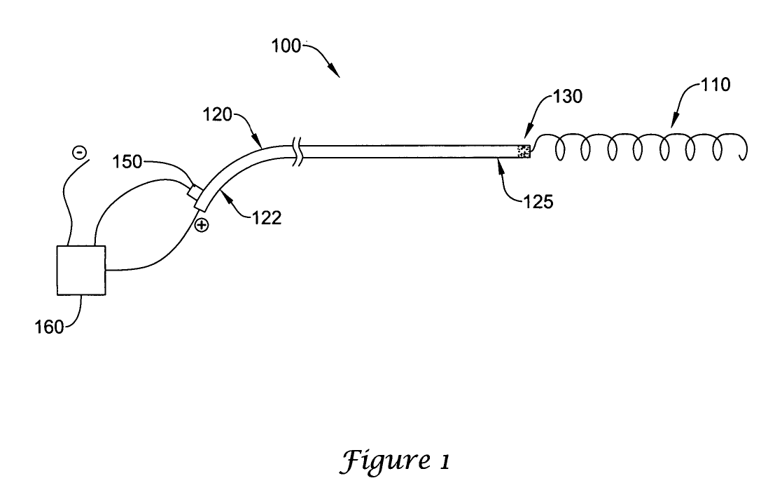

Refer now to FIG. 1, which shows a side, fragmented, cross-sectional view of an example medical device and/or assembly 100 including an elongated member 120, such as a delivery or pusher wire, having a distal end 125 and a proximal end 122. A detachable member 110 is disposed or attached to the elongate member 120 at the distal end 125 in such a manner that the detachable member 110 can be selectively detached from the elongate member 120 through the use of a combination of both electrolytic and mechanical detachment techniques. For example, the detachable member 110 can be disposed or attached to the elongate member 120 such that a detachment zone 130 (e.g. a detachment point, area, region, or structure) is defined. The detachment zone 130 can include structure and/or materials such that when a combination of electrolytic energy and mechanical detachment energy is applied, the detachable member 110 can be detached from the elongate member 120 at the detachment zone 130. The detachment zone 130 can be defined by and/or include a portion of the detachable member 110, a portion of the elongate member 120, or both, and/or may include additional structure. In any case, the detachment zone 130 defines the area where selective separation or detachment of the detachable member 110 from the elongate member 120 can occur when the combination of electrolytic and mechanical detachment energies are sequentially and/or simultaneously applied.

The elongated member 120 can include any of a broad variety of structures or materials. For example, the elongate member 120 can be any generally elongated element having a solid or hollow cross section, having a suitable size or shape, and including materials sufficient to function as a delivery device for the detachable member 110 to the target location within the patient's anatomy. The elongate member 120 may have a generally constant outer diameter, or may include one or more tapers, transitions, shoulders, or other variations in the outer diameter or other geometry to achieve desired characteristics, such as flexibility, pushability, torque transmission, or other such characteristics, as desired. For example, the distal most 10 cm, 20 cm or 30 cm of the delivery wire 120 may be tapered to a smaller diameter. In some examples, the elongated member 120 may be a delivery or pusher wire that may have a diameter in the range of about 0.010 inch to about 0.020 inch (0.254-0.508 mm) and that may have a length in the range of about 50 cm to about 300 cm.

Some examples of suitable materials for elongate member 120 include metals, metal alloys, polymers, or the like, or combinations or mixtures thereof. Some examples of suitable metals and metal alloys include stainless steel, such as 304V, 304L, and 316L stainless steel; nickel-titanium alloy such as linear elastic or superelastic (i.e. pseudoelastic) nitinol; nickel-chromium alloy; platinum or platinum alloys; nickel-chromium-iron alloy; cobalt alloy; tungsten or tungsten alloys; alloys such as Unified Numbering System alloy R30035 (e.g. MP35-N); or the like; or other suitable material, or combinations or alloys thereof, or any other material having a sufficient degree of conductivity and/or other characteristics, as desired. At least a portion of elongate member 120 may be coated with a lubricious polymeric layer, or the like, thus reducing friction during delivery and/or providing an insulative barrier to elongate member 120. Some exemplary embodiments of delivery wires are disclosed in U.S. Pat. Nos. 4,994,069; 5,122,136; 5,234,437; 5,250,071; 5,261,916; 5,304,195; 5,354,295; 5,540,680; 5,569,245; 5,624,449; 5,911,717; 6,022,369; 6,346,091; and 6,463,317; all of which are herein incorporated by reference in their entirety.

The detachable member 110 can include and/or be made of any of a broad variety of structures and/or materials—depending at least somewhat upon its desired function and/or use. For example, the detachable member 110 may be an occlusive member and/or device, for example for use in occluding blood flow and/or creating scar tissue in an aneurysm. In other embodiments, however, the detachable member 110 may be any of a broad variety of other detachable structures, such as a stent, a filter, a tubal block for use in a body passageway, for example, a fallopian tube—to block passage of eggs, or detachable elements containing drugs for delivery to a target site, or other such structure or devices that are intended to be delivered into, and for at least a predetermined time period, remain within the anatomy of a patient after delivery. Any of a broad variety of structures for such devices and/or members can be used.

In some embodiments, the detachable member 110 may be a distal part of the elongated member 120, in that it may be monolithically or integrally formed with the elongated member, but that may also be selectively detached from the remainder of the elongated member 120, for example at a detachment zone 130. In other embodiments, the detachable member 110 may be a separate member and/or structure that is attached or coupled to the distal end of the elongate member 120, and is selectively detachable from the elongate member 120 at a detachment zone 130. In either case, the detachable member 110, once detached from the elongate member 120, may be a separate member or device.

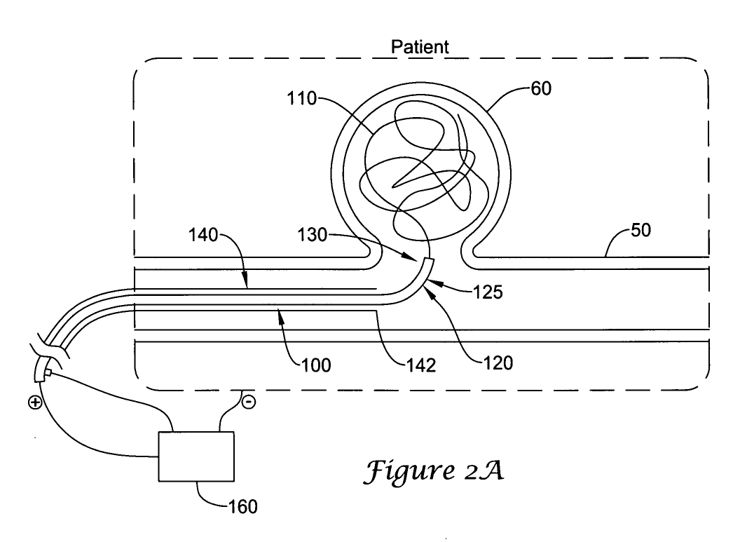

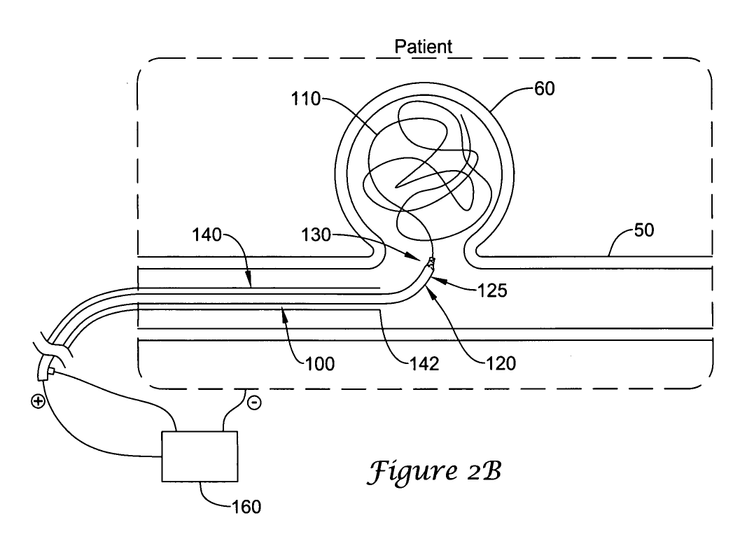

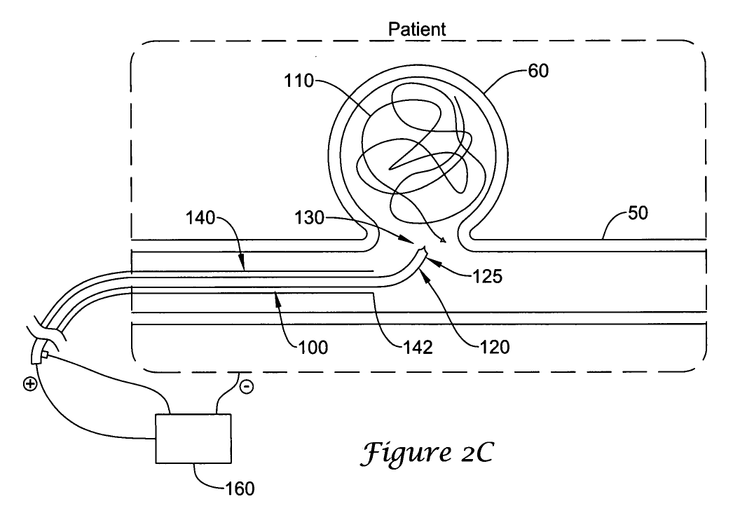

In FIG. 1, the detachable member 110 is shown as an occlusion member, for example, an embolic occlusion device, such as coil, or the like. In some embodiments, the detachable member 110 may be in the range of about 1 to about 50 cm in length, and may have a sufficient flexibility such that the detachable member 110 is capable of deforming and folding and/or bending within a vascular cavity. The detachable member 110 may be extremely pliable and its overall shape may be easily deformed. For example, when inserted within a catheter, occlusion member 110 may be easily straightened to lie axially within the lumen of the catheter. Once disposed out of the distal tip of a catheter, detachable member 110 may convert into a more shapely, nonlinear form, such as the generally helical shape shown in FIG. 1, and may be loosely deformed to the interior shape of a vascular cavity. For example, the detachable member 110 may have a primary coiled and/or helical structure, such as that shown in FIG. 1, in addition to the secondary coiled or tangled configuration when disposed within a target location, such as an aneurism 60, as will be discussed below with reference to FIG. 2. In some embodiments, the formation of a detachable member 110 with desired properties can be accomplished using techniques such as heat treating, use of shape memory materials, or other methods known in the art.

Some examples of suitable materials for detachable member 110 include metals, metal alloys, polymers, or the like, or combinations or mixtures thereof. For example, detachable member 110 may comprise stainless steel, such as 304V, 304L, and 316L stainless steel, or other stainless steel alloy, a nickel-titanium alloy such as linear elastic or superelastic (i.e. pseudoelastic) nitinol, platinum, a platinum alloy, nickel-chromium-iron alloy, cobalt alloy, tungsten or tungsten alloys, alloys such as Unified Numbering System alloy R30035 (e.g. MP35-N), or the like, or any other materials having desired characteristics and/or properties. Platinum or a platinum alloy may be chosen due to the understanding that platinum is not generally subject to oxidation and dissipation during an electrolytic process. The detachable member 110, or a portion thereof, may be coated with a thrombogenic agent, a drug or medication, a biological agent, or other coating. Some exemplary embodiments of detachable members are disclosed in U.S. Pat. Nos. 4,994,069; 5,122,136; 5,234,437; 5,250,071; 5,261,916; 5,304,195; 5,354,295; 5,540,680; 5,569,245; 5,624,449; 5,911,717; 6,022,369; 6,346,091; and 6,463,317; all of which are herein incorporated by reference in their entirety.

The detachable member 110 can include a different material than the elongate member 120. In one embodiment, the detachable member 110 includes a platinum coil and the elongate member 120 includes a stainless steel wire. In other embodiments, the elongate member 120 may include a nickel titanium alloy or platinum wire. However, as discussed above, and as will be understood by those of skill in the art and others, any of a broad variety of materials and/or structures may be used, depending upon the desired characteristics and use of the medical device and/or assembly 100.

The detachment zone 130 may take a variety of shapes, forms, or structures, and/or may include a variety of materials so long as it is designed to rupture, break, disintegrate, or separate when the combination of electrolytic energy and mechanical vibrational energy are applied, as will be discussed in more detail below. The detachment zone 130 can be positioned and/or defined between the detachable member 110 and the rest of the elongate member 120. The detachment zone 130 may be a portion of the elongate member 120, a portion of detachable member 110, may include additional separate and/or discrete structures, and/or any other portion of the assembly 100 which may be oxidized and/or dissipated during an electrolytic process which will weaken the attachment between the detachable member 110 and the elongate member 120. The detachment zone 130 may also include structure and/or material that may be ruptured, broken, or otherwise be made to achieve detachment when mechanical vibrational energy is applied thereto, and in at least some embodiments, is more susceptible to achieve detachment by mechanical vibrational energy after at least a portion is electrolytically degraded to weaken the detachment zone 130.

For example, in some embodiments, the detachment zone 130 may be considered to be defined in a distal portion of the elongated member 120 and/or a proximal portion of the detachable member 110, and may include a material that can be oxidized, dissipated, and/or weakened during an electrolytic process. Some examples of suitable materials may include metals, for example stainless steel, a stainless steel alloy, or the like, or any other material which may be oxidized, dissipated, and/or weakened during an electrolytic process. Additionally, the structure and/or material of the detachment zone 130, once weakened, can be adapted and/or configured rupture, break, or otherwise achieve detachment when mechanical vibrational energy is applied thereto. Other portions of the device 100 separate from the detachment zone 130, such as the remainder of the elongated member 120 and/or detachable member 110, may not be as affected by the electrolytic process and/or mechanical vibrational energy, for example, due to their structure, material, and/or the inclusion of a insulative coating, such as an outer polymeric coating or covering.

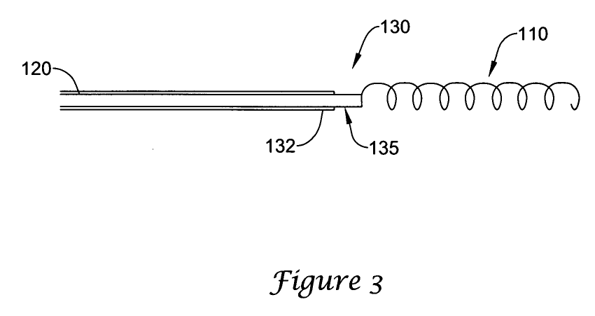

For example in the embodiment shown in FIG. 3, the elongate member 120 may be covered by an insulating material 132. One example of an insulating material is a Teflon lamination. The detachment zone 130 includes a region 135 of the elongate member 120 that is not insulated and is therefore susceptible to electrolytic degradation. The detachable member 110 may be made of a material not susceptible to electrolytic disintegration in blood, such as platinum. In other embodiments, the detachable member 110 can also be insulated. For example, the entire elongate member 120 and detachable member 110 can be made of stainless steel, but the entire structure, except for the detachment zone 130 may be insulated.

In some examples, the detachment zone 130 may include and/or define other and/or additional structure, such as sacrificial coupling structures and/or materials. Some examples of such structures may include a coil, wire, ribbon, sleeve, slot, cut, groove, reduced diameter portion, reduced mass portion, solder, adhesive, crimp, tack, mechanical attachment, friction fit, or the like, that can be adapted and/or configured to couple the detachable member 110 and the elongate member 120, and also may be oxidized, dissipated, and/or weakened during an electrolytic process, and then ruptured, broken, or otherwise made to achieve detachment through the use of mechanical vibrational energy. Some examples of such additional structure will be discussed further below.

With reference still to FIG. 1, the device and/or assembly 100 may also include, or have coupled or attached thereto, one or more power supply 160 for providing the electrolytic and/or mechanical vibrational energy to the assembly 100. A single power supply, for example 160, can be adapted to provide both the electrolytic and mechanical energy, but it should be understood that in other embodiments, separate power supplies may be used. The power supply 160 may be permanently or removably coupled to the proximal end 122 of the elongate member 120. Power supply 160, for example, may be a direct current power supply, an alternating current power supply, or a power supply switchable between a direct current and an alternating current.

So that it can provide the electrolytic energy, the power supply 160 may be electrically coupled to a proximal portion of the elongated member 120. For example, as shown in FIG. 1, a positive terminal of a direct current power supply may be electrically coupled to the proximal portion of the elongated member 120. The power supply 160 may also include a negative terminal that may be coupled to a second electrode member that is adapted and/or configured to be disposed on, within, in contact with, or otherwise in electrical communication with the patient to complete the circuit. Any of a wide variety of structures may be used as the negative electrode, or cathode. For example, a skin electrode, a catheter electrode, a hypodermic electrode, or the like may be used as the cathode. Some example configurations for electrodes that may be used are disclosed in U.S. Pat. Nos. 5,122,136; 5,354,295; 5,540,680; 5,569,245; and 5,624,449, all of which are herein incorporated by reference in their entirety. Some examples of where a portion of a delivery catheter is used as an electrode a disclosed in U.S. patent application Ser. No. 11/148,586 filed on Jun. 9, 2005, which is also herein incorporated by reference in its entirety. Power supply 160 may provide an electrical current through the assembly 100 to initiate an electrolytic process during use of the assembly 100 in a patient, wherein the detachment zone 130 would act as a first electrode, or anode. For example, the detachment zone 130 may be disposed within a fluid medium within the patient, such as a bloodstream, and the bloodstream and/or other tissue within the patient may act as a conducting fluid or medium, completing the electrolytic circuit between the anode and cathode. The power supply 160, such as an alternating or direct current power supply may also be used to initiate an electrothrombosis process, if desired.

To provide the mechanical vibrational energy, the power supply 160 may include or be electrically coupled to a device or structure that provides mechanical vibrational energy to the proximal portion of the elongated member 120. For example, a transducer 150 may be disposed on a proximal portion of the elongated member 120. The transducer can be adapted and/or configured for converting electrical signals from the power supply 160 to mechanical vibrational energy that can be applied to the elongated member 120. The power supply 160 may be electrically coupled to the transducer 150 to provide the electrical signals. The power supply 160 may also include and/or have attached thereto a frequency generator for providing waveforms, and may include a power amplifier to amplify the periodic waveform produced by the frequency generator, which are then delivered to the transducer to create and transfer the mechanical vibrational energy to the elongated member 120. In some embodiments, an ultrasound transducer is used, and the mechanical vibrational energy provided by the transducer is ultrasonic vibrational energy. The frequency and amplitude of the vibrational energy, for example the ultrasound signal, may be selected to produce high stress in the detachment zone 130, fatiguing the elongate member 120 so that it breaks, ruptures, or otherwise separates at the detachment zone 130, leaving the detachable member 110 in the anatomy. In some embodiments, the vibrational energy, such as the ultrasound signal, can be observed on a spectrum analyzer.

The vibrational energy can be transmitted down the elongated member 120 in two different modes of propagation: axial or torsional. In the axial mode, the elongated member 120 may be alternately placed in tension and compression along the axis of the elongated member 120 as the wave travels. In the torsional transmission mode, the elongated member 120 is alternately placed in clockwise and counterclockwise torsion about its axis as the wave travels. Both modes can be useful. A discussion and description of structures and methods of using vibrational energy for detaching a detachable member can be found in U.S. Pat. Nos. 6,022,369 and 6,346,091, which are incorporated herein by reference in their entirety.

FIGS. 2A-2C further illustrate an exemplary method of using the device 100, for example, in delivering the detachable member 120 to a target location 60 within the anatomy of a patient. For example, the device 100 may be used to deliver the detachable member 110 to a target location 60, such as an intravascular aneurysm, to provide and/or aid in the formation of an occlusion within the aneurism 60. However, as discussed above, this method and these particular structures are provided by way of example only, and it should be understood that a wide variety of other uses and/or devices are contemplated.

As shown in FIG. 2A, a delivery device 140, such as a guide or delivery catheter or the like may be advanced through a body passage 50, such as a blood vessel 50, to a location proximate the target location 60, such as an intravascular aneurysm. The distal end 142 of catheter 140 may be positioned such that it mates with, abuts, or extends into the opening of aneurysm 60, or distal end 142 of catheter 140 may remain in vessel 50 proximate the aneurism 60. Using the catheter 140 as a guide, detachable member 110 and the elongated member 120 may be advanced through lumen of the catheter 140 to the target location 60. The elongated member 120 may be further advanced distally, thus urging the detachable member 110 out of the lumen of the catheter 140 and into the interior cavity of aneurysm 60. Unconstrained by the catheter 140, the detachable member 110 may assume a coiled or expanded configuration and may be loosely deformed to the interior shape of the aneurysm 60. Thus, the detachable member 110 may be multiply folded or bent upon itself in the aneurysm 60 to at least partially fill and/or pack the interior of aneurysm 60.

The positive terminal of the power supply 160 is electrically coupled to the proximal portion of the elongated member 120, and the negative terminal is electrically coupled to the patient using an electrode structure, as discussed above. An electrical current may be applied to the elongated member 120 from the power source 160 exterior of the body. Applying a current may initiate an electrolytic process within the detachment zone 130 of the assembly 100. As indicated above, the detachment zone 130 may serve as a first electrode, or anode, and the electrode structure coupled to the negative terminal and patient may serve as a second electrode, or cathode. The bloodstream, and/or other anatomy within the patient, may act as an electrolyte, allowing electrical current to pass through. Thus, a complete electrolytic cell is created. Some additional example methods and/or structures used in electrolytic degradation are disclosed in U.S. Pat. Nos. 5,122,136; 5,354,295; 5,540,680; 5,569,245; and 5,624,449, all of which are herein incorporated by reference in their entirety.

With reference to FIG. 2B, as the electrical current is applied, the electrolytic process take place and oxidation occurs at the anode, which is within the detachment zone 130 of the assembly 100. As such, at least portions of the detachment zone 130 may be oxidized and dissipated—resulting in the loss of mass and/or the weakening of the connection between the elongate member 120 and the detachable member 110.

Additionally, and in combination with and/or after the electrolytic weakening of the detachment zone 130, mechanical vibrational energy is applied thought the elongate member 120 to the weakened detachment zone 130 to aid and/or complete the detachment process. For example, as indicated above, the power supply 160 may provide waveforms through a frequency generator, and a power amplifier may amplify the periodic waveform produced by the frequency generator, which is then delivered to the transducer 150 which creates and transfers the mechanical vibrational energy to the elongated member 120. In some embodiments, the mechanical vibrational energy can be applied during and/or after the electrolytic process. If mechanical energy is applied after the electrolytic process, the time delay can be from a fraction of a second to a few seconds, but is generally less than a minute. For example, the time delay may be in the range of about 0.5 seconds to about 1 minute, or in the range of about 1 second to about 20 seconds. However, other embodiments are contemplated where the time delay may be longer. The application of the mechanical vibrational energy to the detachment zone 130, which has grow weaker through electrolytic degradation, results in the rupture and/or breakage of at least a portion of the detachment zone 130 thereby achieving and/or completing the detachment.

As shown in FIG. 2C, after the detachable member 110 is decoupled from the elongate delivery member 120, through the use of a combination of electrolytic and mechanical vibrational techniques, the detachable member 110 is disposed within the aneurysm 60. The application of the electrolytic and mechanical vibrational energies can be discontinued, and the elongate delivery member 120 and/or catheter 10 may be withdrawn from the vasculature. It may be necessary or desirable in some procedures to dispose one or more additional detachable members 110 within an aneurysm 60 to more fully fill or pack the interior space of the aneurysm 60. In such instances, the catheter 140 may remain positioned in the vasculature while the elongate delivery member 120 is withdrawn and another elongate delivery member 120 including a second detachable member 110 is advanced to the aneurysm 60. The detachment process, including the use of a combination of both electrolytic and vibrational energy may be repeated with the second or subsequent disposition of a detachable member 110 within aneurysm 60. An aneurysm 60 packed with one or more detachable member 110 may allow thrombus to form and/or within cause scarring in the aneurysm 60 to fill the aneurysm the aneurysm 60 thus sufficiently occluding aneurysm 60.

The use of both electrolytic and mechanical vibrational techniques to achieve detachment and/or separation can provide a significant synergistic effect, wherein detachment of the detachable member form the elongated member 120 can be achieved in a rapid and reliable fashion. For example, in at least some embodiments, the use of a combination of electrolytic and mechanical vibrational detachment techniques may achieve significantly better results in terms of timing and reliability that when either technique is used alone.

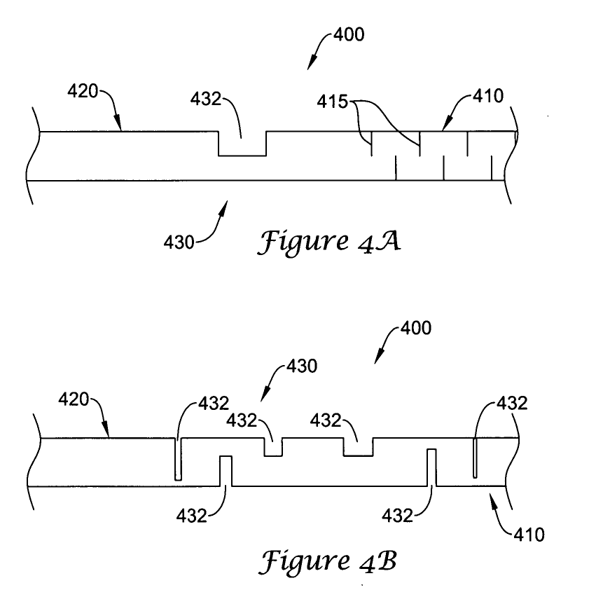

Refer now to FIG. 4A, which shows an alternative embodiment of a medical assembly 400 that may be similar in many respects to the embodiments discussed above, but having alternative constructions and/or structures for the detachment zone and/or detachable member. The medical assembly 400 includes an elongated member 420, such as a hollow or solid delivery member or wire. The elongated member 420 includes and/or forms a detachable member 410 disposed and/or attached on the distal end thereof. A detachment zone 430 is defined and/or disposed between the elongated member 420 and the detachable member 410, and includes one or more notches 432, or reduced thickness portions, defined therein. The notches 432 may provide a region of electrolytic susceptibility, and when weakened by electrolytic degradation, may serve as the detachment zone 430 for rupturing, through fatigue, when vibrational energy is applied to the elongate member 420. As such, applying a combination of electrolytic energy and mechanical vibrational energy to the detachment zone 430, for example using a method similar to that discussed above, can result in detachment of the detachable member 410 from the elongated member 420 at detachment zone 430.

As can also be appreciated, FIG. 4A shows an alternative construction for the detachable member 410. The detachable member 410 in FIG. 4A is a generally elongated member having a plurality of cuts 415 defined in the surface thereof. The cuts 415 may, for example, be made to extend generally transversely into the longitudinal axis of the detachable member 410. For example, the detachable member 410 may be a generally tubular member having a generally circular cross sectional shape and having the plurality of cuts 415 formed therein that may or may not extend through the wall of the tubular member. The cuts 415 may be provided to allow the detachable member 410 to have certain characteristics, such as flexibility characteristics, for example, such that the member 410 can be bent or shaped into a desired configuration within a target location. For example, the cuts 415 may provide the detachable member 410 with flexibility that would allow it to be disposed in an aneurism in a bent, tangled, coiled, or other desirable configuration. Also, the cuts 415 may allow for more surface area exposure to blood for inducing clotting.

FIG. 4B shows another alternative embodiment of a medical assembly 400 that may be similar in many respects to the embodiment discussed above, but includes a plurality of notches 432 that are spaced longitudinally from one another within the detachment zone 430. The notches may be rotated circumferentially with respect to one another. For example, some of the notches 432 may be rotated 90 degrees with respect to each other. Again, the notches 432 may provide regions of electrolytic susceptibility, and when weakened by electrolytic degradation, one or more may serve as the detachment point for rupturing, through fatigue, when vibrational energy is applied to the elongate member 420. Furthermore, the plurality of longitudinally spaced notches 432 may allow for selective detachment to provide a detachable member 410 of particular length and/or may allow for detachment of a plurality of detachable members 410. Each of the notches 432 can be formed or “tuned” with a predetermined depth, width, and/or spacing such that when weakened through electrolytic techniques, they may rupture in response to different amplitudes and frequencies of vibrational energy. In this manner, the user can selectively apply vibrational energy to the elongate member 420 to cause a selected one or more of the notches 432 to rupture. Such rupturing can take place successively to deposit lengths of detachable members 410 at different locations in a body passageway or to deposit all of the lengths, for example to serve as emboli, at a single location. The “tuning” of the notches 432 is both a function of the characteristics of the notches, such as width and depth, and also of the segment lengths between the notches. Such timing could be used to “deposit” great numbers of particles such as for arteriovenous malformation (AVM) therapy.

In order to cause the elongate member 420 to separate at one of the selected notches 432, electrolytic energy is applied, as discussed above, to weaken the detachment zone 430, and a mechanical vibrational energy wave is applied to the elongate member 420 so that the nodal point of the wave falls at a desired notch or notches. A vibrational energy wave causes mechanical resonance in the longitudinal direction where nodal points fall at locations which are spaced every one half of the wavelength. At the nodal points along the wire, the velocity or movement of the wire may be minimal, but the stress may be maximal. Consequently, by applying a vibrational energy wave to the detachment zone 130 through the elongate member 420, the detachment zone 130 would be caused to separate at that location where the greatest stress is occurring and the detachment zone 130 is weakest. In the manner described, appropriate mechanical energy waves can be applied to elongate members such as wires to cause separation at selected notches along the wire. The notches 432 may create spring elements to isolate the intermediate uncut sections of the wire elongate member 420 which have a mass. When a vibrational energy wave at the resonant frequency of the spring/mass system is applied to the wire 420, the wire is excited longitudinally and the sections of mass between the notches vibrate longitudinally at high amplitude which fatigues the spring elements (location of notches) causing them to break.

The detachment zones shown and described above may be configured for detachment in the axial mode. However, torsional waves may be advantageous when using a section of wire which has been provided with cuts and/or notches to enhance its lateral flexibility. The cuts and/or notches may reduce the axial stiffness of the wire, particularly of hollow wire, but can be made in such a way as to not reduce the torsional stiffness of the wire as much. The cut and/or notched wire therefore may be more capable of transmission of torsional than of axial vibration. Where selective detachment is desired, the wave may be required to travel through sections of cut and/or notched wire to reach the various detachment sites. Consequently, the torsion mode may be preferred for selective detachment of cut and/or notched wire.

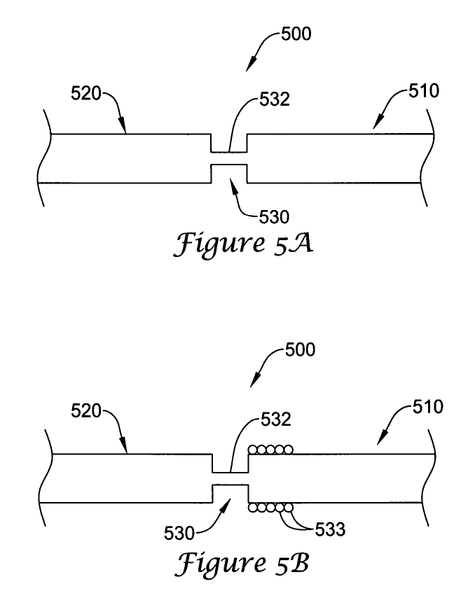

FIG. 5A shows a partial, side, cross-sectional view of another embodiment of a medical assembly 500 that may be similar in many respects to the embodiments discussed above, but having alternative constructions and/or structures for the detachment zone 530. The assembly 500 includes an elongate member 520 and a detachable member 510 disposed and/or attached on the distal end thereof. A detachment zone 530 is defined and/or disposed between the elongated member 520 and the detachable member 510, and includes an annular cut, notch, groove and/or reduced diameter section 532. The annular structure 532 may provide a region of electrolytic susceptibility, and when weakened by electrolytic degradation, may serve as the detachment zone 530 for rupturing, through fatigue, when vibrational energy is applied to the elongate member 520. As such, applying a combination of electrolytic energy and mechanical vibrational energy to the detachment zone 530, for example using a method similar to that discussed above, can result in detachment of the detachable member 510 from the elongated member 520 at detachment zone 530.

FIG. 5B shows an embodiment similar-to that shown in FIG. 5A, wherein like reference numbers indicate similar structure. In FIG. 5B, however, a coil mass 533 may be coiled around the elongate member 520 and/or detachable member 510. In some cases, structure such as the coil mass 533 may further exaggerate the detachment zone 530 when mechanical vibrational energy is applied thereto.

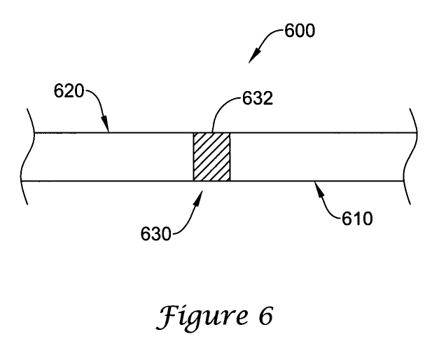

FIG. 6 shows a partial side view of another embodiment of a medical assembly 600 that may be similar in many respects to the embodiments discussed above, but having alternative constructions and/or structures for the detachment zone 630. The assembly 600 includes an elongate member 620 and a detachable member 610 disposed and/or attached on the distal end thereof. The detachable member 610 may be attached to the distal end of the elongate member 620 by a section 632 that may be made of or include material that may be different from the material of the elongate member 620, the detachable member 610, or both. The section 632 of differing material may form the detachment zone 630. For example, section 632 may simply be a section of differing material incorporated into the structure of the assembly 600 to define the detachment zone 630. In some embodiments, the detachable member 610 may be attached to the distal end of the elongate member 620 by a section of attachment material, such as solder, adhesive, brazing material, welding material, or the like—and such material may form the section 632. Such attachment materials may be selected from materials that may be subject to electrolytic degradation and/or that may be somewhat brittle, such as sodium silicate adhesive, such that when a vibrational signal is applied to the elongate member 620, the section 632 may be more susceptible to rupture and achieve detachment of the detachable member 610.

In other embodiments, the section 632 of differing material may be a section of the elongate member 620, the detachable member 610, or both, that was initially of a similar material, but that has been treated to give the section 632 different properties. For example, the section 632 can be heat-treated, H+ embrittled, chemically-treated, such as by etching, or otherwise treated or worked to make the section 632 more brittle, weaker, and/or more susceptible to electrolytic degradation. In a further embodiment, the detachment zone 630 can be a spot weld joining the elongate member 620 to the detachable member 610. The process of spot welding heats the wire, making it more susceptible to fatigue and breaking.

In any respect, the section 632 may provide a region of electrolytic susceptibility, and when weakened by electrolytic degradation, may serve as the detachment zone 630 for rupturing, through fatigue, when vibrational energy is applied to the elongate member 620. As such, applying a combination of electrolytic energy and mechanical vibrational energy to the detachment zone 630, for example using a method similar to that discussed above, can result in detachment of the detachable member 610 from the elongated member 620 at detachment zone 630.

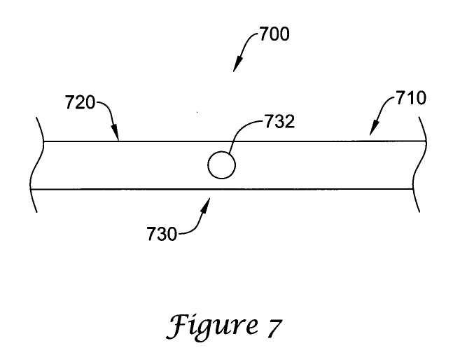

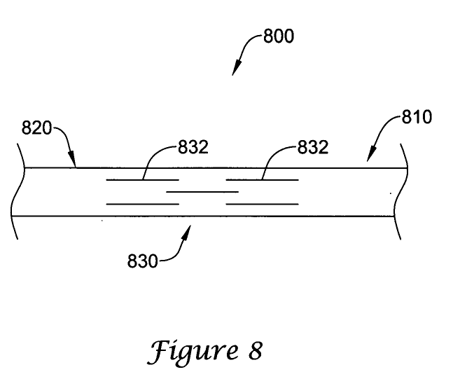

FIG. 7 shows a partial side view of another embodiment of a medical assembly 700 that may be similar in many respects to the embodiments discussed above, but having alternative constructions and/or structures for the detachment zone 730. The assembly 700 includes an elongate member 720 and a detachable member 710 disposed and/or attached on the distal end thereof. The detachment zone 730 includes one or more aperture 732 formed therein, which may provide mechanical weakening of the structure within the detachment zone 730. In any respect, the detachment zone 730 may provide a region of electrolytic susceptibility, and when further weakened by electrolytic degradation, may be ruptured, through fatigue, when vibrational energy is applied to the elongate member 720. As such, applying a combination of electrolytic energy and mechanical vibrational energy to the detachment zone 730, for example using a method similar to that discussed above, can result in detachment of the detachable member 710 from the elongated member 720 at detachment zone 730.

FIG. 8 shows a partial side view of another embodiment of a medical assembly 800 that may be similar in many respects to the embodiments discussed above, but having alternative constructions and/or structures for the detachment zone 830. The assembly 800 includes an elongate member 820 and a detachable member 810 disposed and/or attached on the distal end thereof The detachment zone 830 includes one or more longitudinal cuts and/or grooves 832 formed therein, which may provide mechanical weakening of the structure within the detachment zone 830. Again, the detachment zone 830 may provide a region of electrolytic susceptibility, and when further weakened by electrolytic degradation, may be ruptured, through fatigue, when vibrational energy is applied to the elongate member 820. As such, applying a combination of electrolytic energy and mechanical vibrational energy to the detachment zone 830, for example using a method similar to that discussed above, can result in detachment of the detachable member 810 from the elongated member 820 at detachment zone 830.

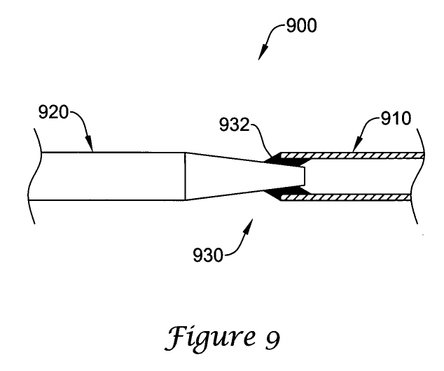

FIG. 9 shows a partial side, cross-sectional view of another embodiment of a medical assembly 900 that may be similar in many respects to the embodiments discussed above, but having alternative constructions and/or structures for the detachment zone 930. The assembly 900 includes an elongate member 920 and a detachable member 910 disposed and/or attached on the distal end thereof. In this embodiment, the detachable member 910 is a structure defining a lumen, such as a tubular structure. The distal end of the elongate member 920 extends into the lumen of the detachable member 910. The detachable member 910 is coupled, attached, and or secured to the distal end of the elongate member 920 by an attachment structure and/or material 932 within the detachment zone 930. For example, the attachment structure and/or material 932 may include solder, adhesive, brazing, or welding material interconnecting the detachable member 910 and the elongate member 920. In other embodiments, the attachment structure and/or material 932 may include alternative structures, such as a coupling coil, wire, ribbon, sleeve, crimp, tack, mechanical attachment, friction fit, or the like. Such attachment structure and/or material 932 may act as sacrificial structure within the detachment zone 930, and when a combination of electrolytic and mechanical vibrational forces are applied thereto, may be designed to degrade and break/rupture to achieve detachment. For example, the attachment structure and/or material 932 may be susceptible to electrolytic degradation when an electrolytic current is applied thereto, and as such may be weakened through electrolytic degradation. When vibrational energy is applied to the so weakened structure 932, it may be ruptured and/or broken, through fatigue. As such, applying a combination of electrolytic energy and mechanical vibrational energy to the detachment zone 930, for example using a method similar to that discussed above, can result in detachment of the detachable member 910 from the elongated member 920 at detachment zone 930.

Naturally, it may be desirable for a user to know immediately when the detachable member of an elongate member has been detached so that the elongate member can be withdrawn from the body passageway. For each of the embodiments described above involving a delivery elongate member portion and one or more detachable member for ultimate detachment at a target site, the combinations of elongate member and detachable members all have natural or resonant frequencies. Thus, when vibrational energy is applied to an elongate member, such as elongate member 120 in FIG. 2, the resonant frequency of the combination of the elongate member 120 and detachable member 110 will have a certain resonant frequency which can be detected by conventional spectrum analysis methods. Consequently, by observing the resonant frequency, a user can obtain instantaneous information as to when the detachable member 110 has detached from the elongate member 120.

In the manner described, a method and apparatus have been provided by which one or more detachable member may be easily, reliably and quickly detached from the delivery portion of an elongate member. The detachable member may be in the form of a coil, mass or other device and may be deposited in vasculature passageways or other body passageways. Mechanical and electrolytic energy are both used to quickly and reliably rupture the detachment zone separating the delivery portion of the wire from the end section.

It is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements.

What is claimed is:

1. A method of disposing a detachable member at a target location in the anatomy of a patient, the method comprising:
providing an elongate member having a detachable member at a distal end, wherein the detachable member is selectively detachable from the elongate member at a detachment zone by the use of a combination of electrolytic and mechanical energy;
inserting the elongate member into the body passageway until the detachable member is disposed at the target location;
delivering current to the elongate member to initiate electrolytic degradation within the detachment zone; and
delivering a mechanical force to the elongate member, thereby detaching the detachable member at the detachment zone; and
withdrawing the elongate member from the body passageway.

2. The method of claim 1, wherein the electrolytic degradation within the detachment zone weakens the detachment zone and makes it more susceptible to detachment when the mechanical vibration is applied.

3. The method of claim 1, wherein the combination of delivering current and delivering mechanical force to the elongate member causes detachment of the detachable member.

4. The method of claim 1, wherein the mechanical force includes vibrational energy.

5. The method of claim 4, wherein the vibrational energy includes ultrasound.

6. The method of claim 1, wherein delivering a mechanical force to the elongate member is performed after delivering current to the elongate member to initiate electrolytic degradation.

7. The method of claim 6, wherein delivering a mechanical force to the elongate member is performed at a predetermined time after delivering current to the elongate member, wherein the predetermined time is in the range of 0.5 seconds to 1 minute.

8. The method of claim 1, wherein delivering a mechanical force to the elongate member is performed during the delivering of current to the elongate member.

9. The method of claim 1, wherein delivering a mechanical force to the elongate member starts at the same time as the start of delivering of current to the elongate member.

10. The method of claim 2, wherein the step of delivering a mechanical force to the elongate member is performed after electrolytic degradation has weakened the detachment zone.

11. The method of claim 1, wherein the detachment zone is made of a material different from the elongate member.

12. The method of claim 1, wherein the detachment zone includes at least one region of reduced cross-section.

13. The method of claim 1, wherein the detachment zone includes a plurality of spaced apart notches.

14. The method of claim 11, wherein the plurality of notches have depths, widths and/or spacings configured to rupture in response to different amplitudes and/or frequencies of vibrational energy, wherein the step of delivering a mechanical force includes applying a particular amplitude and/or frequency of vibrational energy to selectively rupture one or more particular notch to detach one or more distal end portions of desired length.

15. The method of claim 1, wherein the elongate member has a power supply in electrical communication therewith, and wherein the step of delivering current comprises activating the power supply to deliver current to the elongate member.

16. The method of claim 1, wherein the elongate member has a power supply in electrical communication therewith, and wherein the step of delivering a mechanical force comprises activating the power supply to deliver the mechanical force to the elongate member.

17. A medical apparatus having a detachable member for delivery to a target location within the anatomy of a patient, the apparatus comprising:
an elongate member having a proximal end and a distal end;
a detachable member disposed at the distal end of the elongate member; and
a detachment zone adjacent the detachable member, wherein the detachment zone is made of a material susceptible to electrolytic degradation, the detachment zone having a structure susceptible to mechanical separation upon the application of a mechanical force after electrolytic degradation of the detachment zone, such that the detachable member is detached from the elongate member by applying both electrical and mechanical forces.

18. The apparatus of claim 17, further comprising a power supply attached to the proximal end of the elongate member.

19. The apparatus of claim 18, wherein the power supply includes an electrical energy generator.

20. The apparatus of claim 19, wherein the power supply includes a transducer that converts electrical signals to mechanical vibration to provide the mechanical force for detaching the detachable member.

21. The apparatus of claim 20, further comprising a frequency generator for producing waveforms, and a power amplifier to amplify and deliver the waveforms to the transducer.

22. The apparatus of claim 17, wherein the detachable member is an embolic coil.

23. The apparatus of claim 17, wherein the detachment zone comprises stainless steel.

24. The apparatus of claim 17, wherein the detachment zone includes at least one rupturable region that after electrolytic degradation ruptures when vibrational energy is applied thereto.

25. The apparatus of claim 20, wherein the detachment zone includes two or more spaced apart rupturable regions, wherein the rupturable regions are configured to rupture in response to different amplitudes and/or frequencies of vibrational energy, wherein the step of delivering a mechanical force includes applying a particular amplitude and/or frequency of vibrational energy to selectively rupture one or more region to thereby detach one or more distal end portions of desired length.

26. The apparatus of claim 25, wherein the rupturable regions are notches cut partially through the elongate member.

27. A device for disposing a detachable member at a target location in a body passageway, the device comprising:
an elongate member having a proximal portion and a distal portion, and a detachment zone interconnecting the proximal and distal portions;
first means for selectively degrading the detachment zone; and
second means for selectively detaching the distal portion, wherein said distal portion is detached by activating both the first and second means for selectively detaching the distal portion.

28. The device of claim 27, wherein the first means is activated by electrical energy.

29. The device of claim 28, wherein the second means for selectively detaching the distal portion is activated by mechanical energy.

30. The device of claim 29, further including means for providing electrical and mechanical energy to detach the distal portion.

31. The device of claim 28, wherein the detachment zone comprises a material susceptible to electrolytic degradation in blood.

32. The device of claim 31, wherein the elongate member is made of insulated stainless steel, with the detachment zone exposed to allow electrolytic degradation.

33. The device of claim 31, wherein the detachment zone includes a soldered connection between the proximal and distal portions.

34. The device of claim 27, wherein the distal portion is configured to be inserted into a body passageway and occlude the body passageway upon detachment from the elongate member.

35. A device configured to be threaded through a body passageway to a target location, the device comprising:
an elongate member having a proximal end and a distal end;
at least one detachable member detachably attached to the distal end of the elongate member;
wherein each detachable member is selectively detached from the elongate member by applying both electrical and mechanical forces.

36. The device of claim 35, comprising a plurality of detachable members each selectively detachable under different conditions.

37. The device of claim 36, wherein the elongate member is a wire and the plurality of detachable members are formed by a plurality of spaced apart cuts extending partially through the wire.

38. The device of claim 37, wherein the spaced apart cuts have depths and widths configured such that selective application of both electrical and mechanical forces results in the selective detachment of one or more of the plurality of detachable members.

39. The device of claim 38, wherein the spaced apart cuts are configured such that selective application of both electrical and mechanical forces results in detachment of a detachable member of predetermined length.
